(19) United States
(12) Patent Application Publication
(10) Pub. No.: US 2025/0049883 A1
(43) Pub. Date: Feb. 13, 2025

(54) ANIMAL, FUNGAL AND MARINE SOURCES OF CGP AND INCREASED CGP CONCENTRATION FOR DISEASE MANAGEMENT AND FOR TREATMENT OF NON-NEUROLOGICAL AND/OR NEUROLOGICAL CONDITIONS

(71) Applicant: The CGP Lab Limited, Auckland (NZ)

(72) Inventor: Jian GUAN, Auckland (NZ)

(21) Appl. No.: 18/721,128

(22) PCT Filed: Dec. 16, 2022
(86) PCT No.: PCT/NZ2022/050172

§ 371 Date: Jun. 17, 2024

(30) Foreign Application Priority Data
Dec. 17, 2021 (NZ) 783638

(51) Int. Cl. A61K 38/12; A61K 9/48; A61K 38/01; G01N 33/68

(52) U.S. Cl. CPC A61K 38/12; A61K 9/48; A61K 38/014; G01N 33/6893; G01N 33/6896; G01N 2333/65; G01N 2410/00; G01N 2800/28; G01N 2800/2814; G01N 2800/2821; G01N 2800/2835; G01N 2800/2871; G01N 2800/50

(57) ABSTRACT

Described herein are improvements relating to IGF-1 function analysis, adjustment and its application in disease management of non-neurological and/or neurological conditions. More specifically, methods relating to the clinical application of cyclic glycine-proline (cGP) and/or cGP/IGF-1 molar ratio as the plasma biomarker for prediction of risk and recovery of non-neurological and/or neurological conditions with IGF-1 dysfunction and the use of a cGP containing animal, marine or fungal based material such as concentrate/extract of hydrolysed bovine collagen and marine collagen, mushroom and seaweed along with plant-based cGPMAX™ for the treatment of same. The methods more accurately measure IGF-1 function in vivo indirectly using cGP and cGP/IGF-1 molar ratio along with a means to adjust and normalise cGP and cGP/IGF-1 molar ratio (and hence active IGF-1 concentration), and specific treatment methods for individuals with a lower or reduction of cGP level relative to a standard set of baseline data. Supplementation of bovine collagen formulated cGPMAX™ effectively improved the sensory function in patients with diabetic neuropathy.

### TECHNICAL FIELD

Described herein are improvements relating to the analysis of IGF-1 function, adjustment and its application in disease management of non-neurological and/or neurological conditions. More specifically, methods relating to the clinical application of cyclic glycine-proline (cGP) biomarker for prediction of risk and recovery of non-neurological and/or neurological conditions with IGF-1 dysfunction and the use of a cGP containing animal, marine and/or fungal based material such as concentrate/extract of hydrolysed bovine and marine collagen, mushroom and seaweed along with cGP encapsulated in soft gel capsules for the treatment of same.

### BACKGROUND ART

Insulin-like growth factor-1 (IGF-1) is a protein produced as an endocrine hormone. It is a primary mediator of the effects (an effector) of growth hormone and stimulates body growth in nearly every cell in the body. In addition to growth effects, IGF-1 can also regulate nerve cells as well as cellular DNA synthesis.

It is also a neurotropic factor and plays a critical role in a variety of neuronal functions such as cognitive function. IGF-1 is produced throughout life, but is lowest in infants (particularly the neonates), increases during adulthood, and declines with age. Measuring IGF-1 function may be a biomarker of cognitive decline with age and in turn be used to aid in knowing whether interventions such as drug treatments may be useful or not and when to commence such treatments. IGF-1 level is low in infants. However, IGF-1 function plays a critical role during infant brain development, which impacts on brain function in later life.

Plasma IGF-1 concentration however has historically been a poor biomarker for IGF-1 function as direct measurement of IGF-1 provides inconsistent results that cannot be used with any degree of confidence. Historical use for example may be to test for IGF-1 in blood as a screening test for growth hormone deficiency and unusual growth patterns but the results are primarily a guide of total IGF-1 and not a specific measure of active IGF-1.

It is understood that inconsistent results from direct IGF-1 measurement may be due to the majority of IGF-1 in vivo being inactive. It is the active portion that is key. In plasma, more than 95% of IGF-1 binds to IGF-binding protein-3 (IGFBP-3) and once bound, the IGF-binding protein-3 prevents activation of the IGF-1 functional receptor. Therefore, when IGF-1 is measured directly, there is no distinction between active and inactive form, and hence no knowledge of the true active amount of IGF-1 in vivo, the active portion being the biomarker of concern.

The ratio of IGF-1/IGFBP-3 may be used as an alternative for indicating unbound, bioactive IGF-1, but in practice, this ratio does not provide clear indications either as the majority of IGFBP-3 does not bind to IGF-1. As may be appreciated, an alternative measure of IGF-1 may be beneficial as a means to track IGF-1 function and hence use this measure as a biomarker of patient health. For example, cyclic glycine-proline (cGP), a metabolite of IGF-1 is neuroprotective through improving/normalising bioavailability of IGF-1 in plasma.

Corresponding to understanding IGF-1 levels may be the desire to then adjust or support a patient's natural immune and repair system by increasing their IGF-1 active portion.

Further, since IGF-1 is intimately linked to a variety of animal functions, one of many being age-related neurodegenerative function, ways to both measure associated changes and treat, ameliorate or accelerate healing in various related conditions may be of use.

The human body has the ability to protect itself from injury and illness, for example our hormone system becomes more active. However, the capability to help us recover is not always effective in order to achieve a better recovery, especially in older people due to the gradual decline of hormones with age. Stroke happens to young and aged populations. While younger stroke patients can make a more rapid and better recovery, the recovery from older patients can be slow and poor. IGF-1 function is also important for stroke recovery. Thus, a reliable biomarker for IGF-1 function in stroke may be used for the prognosis of a stroke or other neurological conditions and even a method of predicting the ability of stroke recovery may help to design clinical managements for individual patients. However, as above plasma IGF-1 or IGF-1/IGFBP-3 ratio is not reliable biomarker. Thus, it has been found useful to use cyclic glycine-proline (cGP) as a biomarker for IGF-1 function to assist with spontaneous recovery of stroke patients and method of treatment of same.

A key function of IGF-1 in blood circulation is maintaining vascular remodelling, a process of producing endothelial cells and forming capillary networks. The function of capillaries is to deliver nutrients to and take metabolic wastes away from tissues (cells), in order to maintain physiological functions. The research outcome has established the evidence that cGP improves vascular remodelling by regulating the function of IGF-1 in vascular diseases, for example, hypertension, stroke and cognitive impairments.

The health benefits of cGP are well known and been identified in nature plants including blackcurrant where the concentrate/extract of blackcurrant anthocyanins (BCA) that are encapsulated in gelatin hard shell, have been utilised for therapeutic effect.

As an age related condition Parkinson's disease (PD) is the second most common neurodegenerative condition. IGF-1 is a neurotrophic factor and plays an essential role in neuronal survival and maintaining brain function. IGF-1 resistance, characterized by an increase of IGF-1 in circulation and/or brain tissues and impairment of IGF-1 function, has been reported to be associated with neurological deficits and cognitive decline in PD and Alzheimer's disease (AD) patients. As noted above, changes in IGF-1 concentration in serum have been used as a biomarker for IGF-1 dysfunction used for monitoring the prognosis and treatment response in PD, though this is still debatable. High consumption of berry-fruits has been reported to be associated with a lower risk of PD, although the mechanism underlying this putative benefit remains unknown.

However, the concentration of cGP in plants is relatively low and increased after being encapsulated with gelatine soft gel/hard shell. The gelatine soft gel/hard shell is an additional source of cGP, but the clinical application of gelatine encapsulated blackcurrant extract may be sufficient for supporting and managing general health, but not ageing related health issues. Nevertheless, it would be useful to obtain cGP from other sources with higher cGP concentrations to ensure effective bioavailability.

Further aspects and advantages of the improvements relating to the analysis of IGF-1 function, adjustment and disease management of non-neurological and/or neurological conditions due to IGF-1 dysfunction derived from increased concentration of cGP derived sources will become apparent from the ensuing description that is given by way of example only.

### SUMMARY

Described herein are methods relating to the clinical application of cGP plasma biomarker for prediction of risk and recovery of non-neurological and/or neurological conditions with IGF-1 dysfunction and the use of cGP containing animal, marine and/or fungal based material such as a concentration/extraction of hydrolysed bovine collagen for the treatment of same. The methods more accurately measure IGF-1 function in vivo indirectly using cGP and cGP/IGF-1 ratio along with a means to adjust cGP and cGP/IGF-1 ratio (and hence active IGF-1 concentration), and specific treatment methods for individuals with a lower or reduction of cGP level relative to a standard set of baseline data.

In a first aspect, there is provided a method of treating non-neurological and/or neurological conditions associated with IGF-1 dysfunction in an animal comprising the steps of:
a) obtaining a biological specimen from the animal;
b) measuring a concentration of cyclic glycine-proline (cGP) as a plasma biomarker for active concentration dependent insulin-like growth factor 1 (IGF-1) bioavailability in the biological specimen;
c) comparing either the measured cGP concentration and/or ratio of cGP concentration to total measured amount of IGF-1 in the biological specimen to a standard to confirm whether or not, in a continuum of results, the measured cGP concentration and/or ratio of cGP concentration to total measured amount of IGF-1 conforms to the relative standard for estimating IGF-1 function of the individual; and
d) administering a therapeutically effective amount of a concentrate/extract of cGP derived from animal, marine, seaweeds, and/or fungal based material to the animal to:

prevent a decrease in concentration of cGP and/or cGP to total measured IGF-1 ratio in an animal; and/or maintain a pre-existing concentration of cGP and/or cGP to total measured IGF-1 ratio in an animal; and/or increase the concentration of cGP and/or cGP to total measured IGF-1 ratio in an animal.

In a second aspect, there is provided a method of predicting a risk of a non-neurological and/or neurological condition with age in an animal utilising cyclic glycine-proline (cGP) as a plasma biomarker with altered IGF-1 function and biomarker guided interventions comprising the steps of:
a) obtaining a biological specimen from the animal;
b) measuring a concentration of cyclic glycine-proline (cGP) as a plasma biomarker for active concentration dependent insulin-like growth factor 1 (IGF-1) bioavailability in the biological specimen at a first set age of the animal, or an initial stage of the neurological condition, or before treatment of an therapeutically effective amount of a concentrate/extract of cGP derived from animal, marine and/or fungal based material to the animal;
c) re-measuring the concentration of cyclic glycine-proline (cGP) plasma biomarker for active concentration dependent insulin-like growth factor 1 (IGF-1) bioavailability in the biological specimen at further set age intervals of the animal or further stage of the non-neurological and/or neurological condition, or after treatment of the therapeutically effective amount of the concentrate/extract of the cGP derived from animal, marine and/or fungal based material to the animal;
d) comparing either the measured cGP concentration and/or ratio of cGP concentration to total measured amount of IGF-1 in the biological specimen at the set age intervals relative to the first set age, or the initial stage of the non-neurological and/or neurological condition with IGF-1 dysfunction, or before the treatment of the therapeutically effective amount of a concentrate/extract of the cGP derived from animal, marine and/or fungal based material to the animal, in a continuum of results, to confirm whether or not there is a change in the measured cGP concentration and/or ratio of cGP concentration to total measured amount of IGF-1 thereby determining whether the animal is at an increased risk of developing a non-neurological and/or neurological condition from cognitive decline relative to a standard set of baseline data, and wherein the above measured ratio is used to select individual patients for cGP treatment and a suitable dosage for the cGP treatment therein.

In a third aspect, there is provided a method of predicting the ability of spontaneous recovery of an animal with a non-neurological and/or neurological conditions with IGF-1 dysfunction utilising cyclic glycine-proline (cGP) as a plasma biomarker for IGF-1 function comprising the steps of:
a) obtaining a biological specimen from the animal;
b) measuring a concentration of cyclic glycine-proline (cGP) plasma biomarker and active concentration dependent insulin-like growth factor 1 (IGF-1) bioavailability in the biological specimen at a baseline of the animal from onset of the non-neurological and/or neurological condition (<72 h); and c) re-measuring the concentration of cyclic glycine-proline (cGP) plasma biomarker and active concentration dependent insulin-like growth factor 1 (IGF-1) bioavailability in the biological specimen at further regular intervals of the animal during recovery,
d) evaluating functional recovery of the animal from the baseline and at further set intervals, and wherein the baseline concentration of CGP from a continuum of data predicts the short term outcome of non-neurological and/or neurological condition recovery of the animal such that a greater baseline cGP concentration, the more positive prognosis for the animal based on the evaluation of functional recovery.

In a fourth aspect, there is provided the use of a concentrate/extract of cGP derived from animal, marine and/or fungal based material in the manufacture of a medicament formulated to:
prevent a decrease in concentration of cGP and/or cGP to total measured IGF-1 ratio in an animal; and/or maintain a pre-existing concentration of cGP and/or cGP to total measured IGF-1 ratio in an animal; and/or increase the concentration of cGP and/or cGP to total measured IGF-1 ratio in an animal.

In a fifth aspect, there is provided the use of a concentrate/extract of cGP derived from animal, marine and/or fungal based material in the manufacture of a medicament formulated for oral administration to ameliorate the effects of and/or treat non-neurological and/or neurological conditions in a patient in need thereof.

In a sixth aspect, there is provided a functional property comprising a therapeutically effective amount of a concentrate/extract of cGP derived from animal, marine and/or fungal based material formulated for administration to an animal to:
prevent a decrease in concentration of cGP and/or cGP to total measured IGF-1 ratio in an animal; and/or
maintain a pre-existing (normal/physiological) concentration of cGP and/or cGP to total measured IGF-1 ratio in an animal; and/or
increase or to normalise the concentration of cGP and/or cGP to total measured IGF-1 ratio in an animal.

In a seventh aspect, there is provided a method for ameliorating the effects of hypertension and/or a stroke; and/or treating hypertension and/or stroke; and/or reducing the symptoms associated with hypertension and/or stroke in a patient in need thereof, wherein the method comprises administering a therapeutically effective amount of a concentrate/extract of cGP derived from animal, marine and/or fungal based material.

In an eighth aspect, there is provided a method for ameliorating the effects of and/or treating Parkinson's disease or the symptoms associated with Parkinson's disease, or complications associated with cognitive impairment in a patient in need thereof, wherein the method comprises administering a therapeutically effective amount of a concentrate/extract of cGP derived from animal, marine and/or fungal based material.

In a ninth aspect, there is provided a method of treating non-neurological and/or neurological conditions associated with IGF-1 dysfunction in an animal comprising the steps of:
a) obtaining a biological specimen from the animal;
b) measuring a concentration of cyclic glycine-proline (cGP) plasma biomarker for active concentration dependent insulin-like growth factor 1 (IGF-1) bioavailability in the biological specimen;
c) comparing either the measured cGP concentration and/or ratio of cGP concentration to total measured amount of IGF-1 in the biological specimen to a standard to confirm whether or not, in a continuum of results, the measured cGP concentration and/or ratio of cGP concentration to total measured amount of IGF-1 conforms to the relative standard for estimating IGF-1 function of the individual; and
d) administering a therapeutically effective amount of a concentrate/extract of a cGP containing animal, marine and/or fungal based material to the animal to:

prevent a decrease in concentration of cGP and/or cGP to total measured IGF-1 ratio in an animal; and/or maintain a pre-existing concentration of cGP and/or cGP to total measured IGF-1 ratio in an animal; and/or increase the concentration of cGP and/or cGP to total measured IGF-1 ratio in an animal.

In a tenth aspect, there is provided a method of predicting a risk of a non-neurological and/or neurological condition with age in an animal utilising cyclic glycine-proline (cGP) as a plasma biomarker with altered IGF-1 function comprising the steps of:
a) obtaining a biological specimen from the animal;
b) measuring a concentration of cyclic glycine-proline (cGP) plasma biomarker for active concentration dependent insulin-like growth factor 1 (IGF-1) bioavailability in the biological specimen at a first set age of the animal, or an initial stage of the non-neurological and/or neurological condition, or before treatment of an therapeutically effective amount of a concentrate/extract of cGP containing animal, marine and/or fungal based material to the animal;
c) re-measuring the concentration of cyclic glycine-proline (cGP) plasma biomarker for active concentration dependent insulin-like growth factor 1 (IGF-1) bioavailability in the biological specimen at further set age intervals of the animal or further stage of the non-neurological and/or neurological condition, or after treatment of the therapeutically effective amount of the concentrate/extract of cGP containing animal, marine and/or fungal based material;
d) comparing either the measured cGP concentration and/or ratio of cGP concentration to total measured amount of IGF-1 in the biological specimen at the set age intervals relative to the first set age, or the initial stage of the non-neurological and/or neurological condition, or before the treatment of the therapeutically effective amount of a concentrate/extract of cGP containing animal, marine and/or fungal based material to the animal, in a continuum of results, to confirm whether or not there is a change in the measured cGP concentration and/or ratio of cGP concentration to total measured amount of IGF-1 thereby determining whether the animal is at an increased risk of developing a non-neurological and/or neurological condition from cognitive decline relative to a standard set of baseline data, and wherein the above measured ratio is used to select individual patients for cGP containing animal, marine and/or fungal based material treatment and a suitable dosage for the cGP containing animal, marine and/or fungal based material treatment therein.

In an eleventh aspect, there is provided the use of a concentrate/extract of a cGP containing animal, marine and/or fungal based material in the manufacture of a medicament formulated to:
prevent a decrease in concentration of cGP and/or cGP to total measured IGF-1 ratio in an animal; and/or maintain a pre-existing concentration of cGP and/or cGP to total measured IGF-1 ratio in an animal; and/or increase the concentration of cGP and/or cGP to total measured IGF-1 ratio in an animal.

In a twelfth aspect, there is provided the use of a concentrate/extract of cGP containing animal, marine and/or fungal based material in the manufacture of a medicament formulated for oral administration to ameliorate the effects of and/or treat non-neurological and/or neurological conditions in a patient in need thereof.

In a thirteenth aspect, there is provided an extract comprising a therapeutically effective amount of concentrate/extract of cGP containing animal, marine and/or fungal based material formulated for administration to an animal to:
prevent a decrease in concentration of cGP and/or cGP to total measured IGF-1 ratio in an animal; and/or maintain a pre-existing (normal/physiological) concentration of cGP and/or cGP to total measured IGF-1 ratio in an animal; and/or
increase or to normalise the concentration of cGP and/or cGP to total measured IGF-1 ratio in an animal.

In a fourteenth aspect, there is provided a method for ameliorating the effects of hypertension and/or a stroke; and/or treating hypertension and/or stroke; and/or reducing the symptoms associated with hypertension and/or stroke in a patient in need thereof, wherein the method comprises administering a therapeutically effective amount of a concentrate/extract of cGP containing animal, marine and/or fungal based material.

In a fifteenth aspect, there is provided a method for ameliorating the effects of and/or treating Parkinson's disease or the symptoms associated with Parkinson's disease, or complications associated with cognitive impairment in a patient in need thereof, wherein the method comprises administering a therapeutically effective amount of a concentrate/extract of cGP containing animal, marine and/or fungal based material.

In a sixteenth aspect, there is provided a method for ameliorating the effects of and/or treating the metabolic disorders and the associated complications, for examples type 2 diabetes-associated peripheral neuropathy, kidney function failure, retinopathy and other complications due to poor function of capillaries.

In a seventeenth aspect, there is provided a method for ameliorating the effects of and/or treating the cancer, including terminal stage cancers.

In a eighteenth aspect, there is provided a soft gel or hard shell capsule used to encapsulate cGP extracted material for administration thereof and increase the cGP concentration of the extracted material contained therein over a period of time.

Advantages of the above may be varied. With respect to a biomarker, cGP as noted is a stable metabolite of IGF-1 and based on the inventor's work, is easily measured in biological specimens taken from an animal. cGP does not have the same variability as IGF-1 in terms of distinguishing active from inactive forms and hence is more reliable than measuring total IGF-1. With respect to the described methods of measuring the changes in cGP and/or addressing neurological conditions, an advantage of the clinical application of cyclic glycine-proline (cGP) as a reliable biomarker allows for selection of suitable patients for treatment and individual dosage regimes. The increase of cGP indicates the risk of a neurological condition and the reduction of cGP indicates the stage of the neurological condition. Advantageously, suitable patients for treatment may be selected based on their cGP level and the changes of cGP levels can be easily monitored for a tailored or personalised treatment dosage regime. It has been advantageously found that high concentrations of cGP in the collagen from animal and marine tissues are a thousand times higher than the plant based material counterparts. The supplementation of higher concentration products derived from animal, marine and/or fungal is extremely advantageous when adjusting the dosage regime as this allows for faster effective treatment of the non-neurological and/or neurological conditions where the products assist the body in reaching the effective concentration faster, thus improving management or supporting the management of acute conditions, such as traumatic brain injury (TBI), stroke and other types of injuries.

The use of cGP derived from animals, marine and/or fungal material in such a measurable and known manner has not been known or completed in the art. The cGP extracted from these sources has not been used in the known and measurable manner now possible and as described herein. The experimental results exhibit a new or at least alternative use for cGP extracts that may provide significant and measurable benefit to patients potentially also without interfering with existing art treatments or causing unwanted side effects. Further, with respect to natural products and associated bioactive compounds generally, the methods and uses described herein provide unique ways to know when it is appropriate to intervene with a treatment and further, provide ways to measure the success or otherwise of a treatment. This insight into timing and efficacy is something many pharmaceutical drugs would aspire to and not something commonly observed in natural products containing bioactive compounds.

A further advantage the inventors have unexpectedly found is that soft gel capsules used to encapsulate cGP extracted material increase the cGP concentration of the extracted material contained therein over a period of time.

### BRIEF DESCRIPTION OF THE DRAWINGS

Further aspects of the methods relating to the clinical application of cyclic glycine-proline (cGP) plasma biomarker for prediction of risk and the recovery of non-neurological and neurological conditions associated with IGF-1 dysfunction and the use of a cGP containing animal, marine and/or fungal based material such as a concentrate/extract of hydrolysed bovine collagen powder for the treatment of same will become apparent from the following description that is given by way of example only and with reference to the accompanying drawings in which:

FIG. 1 shows the relative concentration of cGP (ng/mg) in animal, fungal, seaweeds and marine products;

FIG. 2 shows that the cGP concentration in urine is significantly increased 3 hours after oral consumption of 100 mg of bovine collagen powder;

FIG. 3 shows a Pearson correlation analysis suggesting a strong positive correlation between urine and plasma cGP concentrations (r=0.78, p<0.001); and

FIG. 4 shows the improved sensory deficit after oral supplementation of new formulation of cGPMAX™ that is blended with bovine collagen. Repeated measure ANOVA suggested the sensory deficits were reduced 3 and 6 months after the supplementation of cGPMAX™ (p=0.0007).

### DETAILED DESCRIPTION

As noted above, described herein are methods relating to the clinical application of cyclic glycine-proline (cGP) plasma biomarker for prediction of risk and recovery of non-neurological and neurological conditions and the use of a cGP-containing animal, marine and/or fungal based material such as concentrate/extract of bovine collagen along with cGP encapsulated in soft gel capsules for the treatment of same. The methods more accurately measure IGF-1 in vivo indirectly using cGP and cGP/IGF-IGFBP3 ratio along with a means to adjust cGP and cGP/IGF-1 ratio (and hence active IGF-1 concentration), and specific treatment methods for individuals with a lower or reduction of cGP level relative to a standard set of baseline data.

For the purposes of this specification, the term ‘about’ or ‘approximately’ and grammatical variations thereof mean a quantity, level, degree, value, number, frequency, percentage, dimension, size, amount, weight or length that varies by as much as 30, 25, 20, 15, 10, 9, 8, 7, 6, 5, 4, 3, 2, or 1% to a reference quantity, level, degree, value, number, frequency, percentage, dimension, size, amount, weight or length.

The term ‘substantially’ or grammatical variations thereof refers to at least about 50%, for example 75%, 85%, 95% or 98%.

The term ‘comprise’ and grammatical variations thereof shall have an inclusive meaning—i.e. that it will be taken to mean an inclusion of not only the listed components it directly references, but also other non-specified components or elements.

The term extract should be understood to be an extract of cGP derived from a raw material where the extract and/or concentrate may be a source or carrier of cGP when suitably extracted from an animal, marine and/or fungal source.

In a first aspect, there is provided a method of treating non-neurological and/or neurological conditions associated with IGF-1 dysfunction in an animal comprising the steps of:
a) obtaining a biological specimen from the animal;
b) measuring a concentration of cyclic glycine-proline (cGP) as a plasma biomarker for active concentration dependent insulin-like growth factor 1 (IGF-1) bioavailability in the biological specimen;
c) comparing either the measured cGP concentration and/or ratio of cGP concentration to total measured amount of IGF-1 in the biological specimen to a standard to confirm whether or not, in a continuum of results, the measured cGP concentration and/or ratio of cGP concentration to total measured amount of IGF-1 conforms to the relative standard for estimating IGF-1 function of the individual; and
d) administering a therapeutically effective amount of a concentrate/extract of cGP derived from animal, marine and/or fungal based material to the animal to:

prevent a decrease in concentration of cGP and/or cGP to total measured IGF-1 ratio in an animal; and/or maintain a pre-existing concentration of cGP and/or cGP to total measured IGF-1 ratio in an animal; and/or increase the concentration of cGP and/or cGP to total measured IGF-1 ratio in an animal.

In a second aspect, there is provided a method of predicting a risk of a non-neurological and/or neurological condition with age in an animal utilising cyclic glycine-proline (cGP) as a plasma biomarker with altered IGF-1 function comprising the steps of:
a) obtaining a biological specimen from the animal;
b) measuring a concentration of cyclic glycine-proline (cGP) as a plasma biomarker for active concentration dependent insulin-like growth factor 1 (IGF-1) bioavailability in the biological specimen at a first set age of the animal, or an initial stage of the neurological condition, or before treatment of an therapeutically effective amount of a concentrate/extract of cGP derived from animal, marine and/or fungal based material to the animal;
c) re-measuring the concentration of cyclic glycine-proline (cGP) plasma biomarker for active concentration dependent insulin-like growth factor 1 (IGF-1) bioavailability in the biological specimen at further set age intervals of the animal or further stage of the non-neurological and/or neurological condition, or after treatment of the therapeutically effective amount of the concentrate/extract of the cGP derived from animal, marine and/or fungal based material to the animal; and
d) comparing either the measured cGP concentration and/or ratio of cGP concentration to total measured amount of IGF-1 in the biological specimen at the set age intervals relative to the first set age, or the initial stage of the non-neurological and/or neurological condition with IGF-1 dysfunction, or before the treatment of the therapeutically effective amount of a concentrate/extract of the cGP derived from animal, marine and/or fungal based material to the animal, in a continuum of results, to confirm whether or not there is a change in the measured cGP concentration and/or ratio of cGP concentration to total measured amount of IGF-1 thereby determining whether the animal is at an increased risk of developing a non-neurological and/or neurological condition from cognitive decline relative to a standard set of baseline data, and wherein the above measured ratio is used to select individual patients for cGP treatment and a suitable dosage for the cGP treatment therein.

In a third aspect, there is provided a method of predicting the spontaneous recovery of an animal with a non-neurological and/or neurological conditions with IGF-1 dysfunction utilising cyclic glycine-proline (cGP) as a plasma biomarker for IGF-1 function comprising the steps of:
a) obtaining a biological specimen from the animal;
b) measuring a concentration of cyclic glycine-proline (cGP) plasma biomarker and active concentration dependent insulin-like growth factor 1 (IGF-1) bioavailability in the biological specimen at a baseline of the animal from onset of the non-neurological and/or neurological condition (<72 h in acute conditions);
c) re-measuring the concentration of cyclic glycine-proline (cGP) plasma biomarker and active concentration dependent insulin-like growth factor 1 (IGF-1) bioavailability in the biological specimen at further regular intervals of the animal during recovery;
d) evaluating functional recovery of the animal from the baseline and at further set intervals, and wherein the baseline concentration of CGP from a continuum of data predicts the short term outcome of non-neurological and/or neurological condition recovery of the animal such that a greater baseline cGP concentration, the more positive prognosis for the animal based on the evaluation of functional recovery.

Cyclic glycine-proline (cGP) is a stable fragment naturally formed by unbound and bioactive IGF-1. cGP competes with IGF-1 for binding of IGFBP-3 in a concentration dependent manner. The inventor has found that cGP is a measurable and reliable plasma biomarker correlated strongly to the amount of active IGF-1 as a result of the concentration dependent manner of inactivation noted above. That is, by measuring cGP concentration, the presence or otherwise of active IGF-1 function can be measured instead of reliance on variable direct IGF-1 measured results.

The inventor's work has shown that a higher concentration of cGP would free more IGF-1 from IGFBP-3 via the concentration dependent manner noted above hence, leading to an increase in bioavailable IGF-1.

In addition to direct cGP measurement, the inventors have also identified that relative concentrations of cGP to IGF-1 (i.e. the cGP/IGF-1 molar ratio) may also represent the amount of bioavailable IGF-1 in a patient, thus may also potentially fulfil the role for a suitable biomarker for IGF-1 associated cognitive function. It also has been found that the increase in cGP indicates the risk of a neurological condition and the reduction of cGP correlates to the stage of the neurological condition. Hence, it is envisaged that that the treatment with cGP extracts may prevent or delay the on-set of cognitive decline.

The term ‘biomarker’ as used herein refers to cGP or the molar ratio of cGP to total IGF-1 traceable in an animal as a means to examine a function of animal health and function. Health and function may comprise but are not limited to detection of a ‘normal’ or ‘healthy’ state for the animal relative to either a historical standard measurement for the animal or for a population. Health and function may also comprise detection of a non-normal or unhealthy animal state perhaps linked with a condition, disease or unusual state. As may be appreciated, terms such as ‘normal’ and ‘healthy’ are subjective terms, however in the context of this specification, the terms are a relative measure of the biomarker compound or ratio noted compared to either historical details about the animal and/or variation to a population referred to as a standard.

As noted above, the standard may be based on a set of data collected for an individual animal. For example, for a period of time-say 1 week to 6 months to 2 years-averaged cGP and cGP to total IGF-1 molar ratio data may be collected for the animal. Variations in the biomarker concentration or ratio may be observed and analysed to find a ‘typical’ figure for the animal and that data and the extent of any deviations used to understand a normal or standard figure versus a typical variation and hence confirm when a non-normal or atypical change occurs.

The standard may instead, or in conjunction with individual animal data, be based on a set of data collected for a population. The population may for example be a gender group, a group defined by age, a group defined by symptom, condition or disease state and so on.

The cGP and/or IGF-1 may be measured in a biological specimen taken from the animal. For the purposes of this specification, the terms ‘biological specimen’, ‘bodily sample’, or ‘sample’ may be used interchangeably and refer collectively to a specimen taken or extracted from a patient, stored and later analysed. Techniques for extraction of the specimen may for example be via swabs, venipuncture, sticks, biopsy, fractionation, urination, stool sample and so on. In selected embodiments, the biological specimen may be: cerebrospinal fluid (CSF), plasma, urine, breast milk, any other biological specimens (tears and any other bodily function) and combinations thereof.

cGP and/or total IGF-1 in the biological specimen may be measured by techniques selected from: ELISA, HPLC, mass spectrometry, and combinations thereof. Other art analysis techniques may also be used and reference to these techniques should not be seen as limiting.

In a fourth aspect, there is provided the use of a concentrate/extract of cGP derived from animal, marine and/or fungal based material in the manufacture of a medicament formulated to:
prevent a decrease in concentration of cGP and/or cGP to total measured IGF-1 ratio in an animal; and/or
maintain a pre-existing concentration of cGP and/or cGP to total measured IGF-1 ratio in an animal; and/or
increase the concentration of cGP and/or cGP to total measured IGF-1 ratio in an animal.

In a fifth aspect, there is provided the use of a concentrate/extract of cGP derived from animal, marine and/or fungal based material in the manufacture of a medicament formulated for oral administration to ameliorate the effects of and/or treat non-neurological and/or neurological conditions in a patient in need thereof.

In a sixth aspect, there is provided a concentrate/extract comprising a therapeutically effective amount of cGP derived from animal, marine and/or fungal based material formulated for administration to an animal to: prevent a decrease in concentration of cGP and/or cGP to total measured IGF-1 ratio in an animal; and/or maintain a pre-existing (normal/physiological) concentration of cGP and/or cGP to total measured IGF-1 ratio in an animal; and/or
increase or to normalise the concentration of cGP and/or cGP to total measured IGF-1 ratio in an animal.

In a seventh aspect, there is provided a method for ameliorating the effects of hypertension and/or a stroke; and/or treating hypertension and/or stroke; and/or reducing the symptoms associated with hypertension and/or stroke in a patient in need thereof, wherein the method comprises administering a therapeutically effective amount of a concentrate/extract of cGP derived from animal, marine and/or fungal based material.

The inventor has found that when applying a concentrate/extract of cGP to a patient with hypertension (high blood pressure), their blood pressure reduces to acceptable base levels.

The non-neurological conditions or diseases for treatment and to predict recovery may be selected from, but not limited to hypertension, acute brain injuries (for example, concussion), weight changes in obese women and post-natal development.

In an eighth aspect, there is provided a method for ameliorating the effects of and/or treating Parkinson's disease or the symptoms associated with Parkinson's disease, or complications associated with cognitive impairment in a patient in need thereof, wherein the method comprises administering a therapeutically effective amount of a concentrate/extract of cGP derived from animal, marine and/or fungal based material.

The neurological conditions or diseases for treatment may be selected from, but not limited to Cerebrovascular accident (CVA) or stroke, Mild Cognitive Impairment (MCI), Alzheimer's, vascular dementia, hypertension and its associated brain complications, Parkinson's and/or any other ageing related conditions or IGF-1 deficiency related conditions.

Stroke is an exemplary neurological condition in which poor blood flow to the brain results in cell death typically resulting in part of the brain not working properly. The inventor has identified via the biomarker noted above that cGP concentration and cGP to total IGF-1 ratio changes in stroke patients and the extent to which the cGP or ratio decreases is a strong indicator of patient recovery and outcomes. Lower decreases are associated with faster recovery and potentially fewer on-going issues. The exact mechanism for this is not proven however, since IGF-1 is associated with growth it follows that a lower cGP level or cGP to total IGF-1 ratio corresponds to less active IGF-1 and hence slower growth of neural pathways hence a slower recovery. Based on this finding, the inventor has shown that administration of a concentrated cGP extract derived from animal, marine and/or fungal based material may increase cGP levels and/or cGP to total IGF-1 ratio hence ameliorating the effects of stroke, treating stroke or at least reducing the symptoms associated with stroke and/or other neurological conditions.

A further example of a neurological condition evaluated by the inventor is Parkinson disease (PD) which is the second most common neurodegenerative condition. As aforementioned, insulin-like growth factor-1 (IGF-1) is a neurotrophic factor and plays an essential role in neuronal survival and brain function. IGF-1 resistance, characterized as increase of circulating IGF-1 with impaired IGF-1 function, plays a role in disease progression of idiopathic PD, cognitive impairments and pathology of PD. Thus, changes of plasma concentration of IGF-1 also have been evaluated by the inventor as a biomarker for monitoring IGF-1 function, in order to predict the prognosis and treatment response in PD and other conditions such as metabolic disorders and cancer.

For the purposes of this specification, the terms ‘amelioration’, ‘treat’ or ‘reduce the symptoms of’ refers to reducing the measured impact of at least one indication or symptom associated with a neurological condition by at least 1, or 2%, or 3%, or 4%, or 5%, or 6%, or 7%, or 8%, or 9%, or 10% compared to that measured in the patient with no blackcurrant extract administration.

Administration may halt a decrease in cGP concentration or decrease in cGP to total IGF-1 molar ratio as a result of a non-normal state such as that caused by disease or a condition or from symptoms associated with a disease or condition. The term ‘halt a decrease’ as used refers to the cGP concentration and/or cGP to total IGF-1 ratio remaining within at least 1, or 2%, or 3%, or 4%, or 5%, or 6%, or 7%, or 8%, or 9%, or 10% of a measured concentration or ratio prior to administration.

Administration may increase cGP concentration and/or cGP to total IGF-1 molar ratio by at least 1, or 2%, or 3%, or 4%, or 5%, or 6%, or 7%, or 8%, or 9%, or 10% above what would be measured in the patient with no cGP extract administration. The inventor has unexpectedly found that there may be a 25% increase of cGP in the cerebrospinal fluid (CSF) after cGP extract supplementation. Without being bound by theory, this suggests effective uptake by the brain following oral administration and is a significant finding for treatment of any brain diseases. Furthermore, the cGP extracts may increase the formation of cGP in human blood plasma. As far as the inventor is aware, there have been no other studies which have shown an effective brain uptake of at least 25% cGP in the CSF. Further clinical trials are in progress to confirm the efficacy and uptake of the supplementation of high concentration cGP products and how these supplements assist the body in reaching the effective concentration faster. Also, elucidation of the mechanism may provide essential scientific evidence to further support cGP extracts as an intervention for normalising IGF-1 function.

The animal referred to in the above aspects may be a human. The animal may alternatively be a non-human animal. Reference to an animal may be used interchangeably herein with the words ‘subject’ or ‘patient’ and reference to one or the other should not be seen as limiting.

In one embodiment, the animal to which the cGP extract is administered may be a healthy animal not exhibiting any non-normal cGP and/or cGP to total IGF-1 ratio. In this embodiment, the extract may be administered proactively as a means to prevent or avoid a change in cGP or cGP to total IGF-1 ratio that may in turn be associated with a disease, condition or symptoms of a disease or condition. Alternatively, the animal may have a pre-existing condition, disease state, and/or symptoms associated with a condition or disease state.

As noted above, terms such as ‘normal’ and ‘healthy’ are subjective terms, however in the context of this specification, the terms are a relative measure of the biomarker compound or ratio noted compared to either historical details about the animal and/or variation to a population referred to as a baseline or standard. The baseline of individual may vary between age, gender, and any other medical conditions that may be associated with IGF-1 function. It has been found that the changes of cGP and/or cGP to total IGF-1 ratio from the baseline of an individual is more critical information for guiding treatments and cGP dosage regimes. An exemplary study conducted by the inventor showed cGP level in plasma to be 3.5 ng/mg in healthy women, 8-10 ng/ml in elderly people 50-70 years of age and 12 ng-15 ng/ml in PD patients due to the decline in IGF-1 function of elderly people and IGF-1 resistance in PD patients. The increase of cGP concentration is an endogenous response to maintain IGF-1 function. When this response loses its effectiveness, patients develop symptoms for example dementia in PD patients.

The medicament described may be formulated in one embodiment for oral administration. Oral administration is a non-invasive and simple means to administer bioactive compounds and a method well explored in terms of oral supplements. In addition, for the purposes of regulatory approvals, it may be a useful approach to make for marketing the method described. Despite reference to oral administration, the medicament could also be formulated for parenteral administration, for example non-limiting examples being as an injection, sublingual wafer or suppository. Overcoming the blood brain barrier (BBB) has been described extensively in the art for many bioactive compounds, hence some digression in whether oral or parenteral methods are preferred or even whether the BBB will allow transfer. The inventor has found that oral administration of the cGP extracts does cross the blood brain barrier and hence may be useful means for administration.

The concentrate/extract itself may in one embodiment be a dried powder. The processed powder may be micronized to a diameter in the micron range and may have a particle size of less than 1000, or 100, or 10, or 1 micron. The medicament comprising the extract may be formulated as a pill, tablet, capsule, liquid, powder, micronized powder, gel, soft gel full of liquid and combinations of these forms. Art extracts may be coarse powders with a particle size greater than 1000 micron. Such extracts can be difficult to solubilise in aqueous environments, hence micronized forms of the extract noted may be useful since they are easier to solubilise and hence more rapidly and more completely ingested and moved to the animal's bloodstream.

As noted above, the dose is a therapeutically effect amount. In one embodiment, a therapeutically effective dose of concentrated cGP may provide a dose of at least between 10,000 to 100,000 ng (10-100 μg) as daily dose. It has been discovered that the high concentration of cGP in collagen from animal and marine tissues can be several thousand times higher than the plant-based equivalent. The blend of ingredients as a formula to provide effective daily doses for the purpose of maintaining health, disease prevention or treatment for neurological or non-neurological conditions. The clinical application of plant based cGP have some limitations due to the low concentration of cGP. For example, it is restrictive for health management of the people with IGF-1 dysfunction requiring an increased demand and uptake of cGP concentration in their body. Thus, the initial period of cGP supplementation derived from plant sources will normally take weeks or months to see the effect for the accumulation of cGP to be at an efficacious level. This is a limitation of plant sourced cGP for its clinical application in disease management, particularly the acute conditions.

Advantageously, the supplementation of higher concentration cGP products derived from animal, marine and/or fungal sources will help the body reach the therapeutically effective concentration faster. This is essential for the management or in supporting the management of acute conditions, such as traumatic brain injury (TBI), stroke, other type of injuries and complications from metabolic disorders such as diabetic peripheral neuropathy (Example 3) and cancer.

As should be appreciated, the dose used may vary depending on factors such as individual animal metabolism, animal species, animal bodyweight, animal age, medical conditions, health status and other factors, hence these doses should not be seen as limiting. Other types of organic based material containing a concentration of cGP and/or combinations thereof could conceivably be used with this invention.

The ingredients with high concentration of cGP may be derived from any animal, marine and/or fungal sources including any living organism, but not limited to bovine, marine and bone collagen, Types 1 to 7 or the like.

The health claims of collagen are mainly limited to skin, hair and nails. Basement membrane (BM) is the part of structure of blood vessels, particularly those in brain. Collagen is the biological and structural element of the BM. Experimental mutation of collagen leads to the dysfunction of BM, which has been demonstrated in studies in animal models of stroke, other cerebral vascular diseases and some developmental neurological conditions. However, these known studies have been limited to examine the role for collagen mutations and their association with pathological changes of tissues and as per the Applicant's invention have made no connections to the health claim and disease management of collagen including neurological and vascular conditions. Furthermore, there is no link or teaching and suggestion of cGP to collagen in the art.

The hydrolysation process cleaves collagen/gelatine proteins and forms linear peptides with repeated sequences of hydrolysed proline-glycine. A cyclisation of these linear peptides leads to production of cGP. Apart from the replacement of collagen/gelatine to tissues, the inventor claims using collagen or gelatine as viable sources for natural cGP in order to normalise IGF-1 function.

In some embodiments, the extract may be hydrolysed bovine collagen powder, collagen marine skin powder, hydrolysed fish skin powder and bone collagen.

The processes described may be produced using body parts selected from: skins, proteins, bones, muscles tendons and the like and/or combinations thereof. In one embodiment, the extract may be produced by maceration and other novel extraction methods from the animal, marine and/or fungal material, followed by drying to form a concentrated powder that may then be micronized. The micronized powder may optionally be encapsulated if a capsule form is desired or otherwise processed to a final form. In another embodiment, extract concentrates and/or liquid products may be a source of the cGP extracted products.

The blend may comprise Cyclic glycine-proline (cGP) in a concentration to provide at least 10,000 ng or more as daily dose and/or at least the required amount of cGP to deliver a health benefit.

Whilst not being bound by theory, it is understood that these extracts, either in combination or separately, may be attributed to the observed effect in vivo on the binding of IGF-1 and hence the observed increase in cGP concentration and/or cGP to total IGF-1 ratio. That said, the extract may comprise other compounds besides cGP and, being derived from an animal, marine and/or fungal source, there may be other bioactive compounds or synergies from the extract not purely attributable to these compounds. As a result, reference to these compounds specifically is not intended to exclude the bioactivity of other compounds possibly present in the extract.

Advantages of the above may be varied. With respect to a biomarker, cGP as noted is a stable metabolite of IGF-1 and based on the inventor's work, is easily measured in biological specimens taken from an animal. cGP is formed from an unbound active form of IGF-1 and hence is more reliable than measuring total IGF-1. With respect to the described methods of increasing cGP and/or addressing neurological conditions, the use of cGP concentrate/extract derived from animal, marine and/or fungal sources in such as measurable and known manner has not been known or completed in the art. In this way, animal products have much higher cGP concentration than that of plant derived cGP such as blackcurrant. It has also been advantageously found that oral administration of bovine collagen may further increase the cGP concentration in a human's system (urine) of participants who had been using cGPMax™ long-term. A disadvantage of the lower concentration of cGP in plant based products, is that a longer ‘build-up’ period is often experienced by the consumers for any improvements to be observed and would not be suitable for treating acute conditions and/or the patients with severe conditions. Thus, the significantly higher cGP concentration of animal, marine and fungal derived products can increase the cGP concentration to efficacious levels much faster than plant-based products. Given the plant based cGPMax™ is primarily used for supporting health of normal people, the other sources with higher concentration of cGP would be suitable for supporting the health of people with medical conditions with IGF-1 deficiency, new products from animal, marine, plant and fungal derivatives may be developed for improving stroke recovery and other medication conditions with systemic and cerebral vascular conditions due to IGF-1 deficiency. Thus, any newly developed products may also be suitable for acute conditions that have increased demand for cGP in their system. For example, traumatic brain injury and on-set of stroke.

The inventor also has found that collagen and gelatine (the heated form of collagen) is high in glycine-hydroxyproline amino acid sequences and unexpectedly the breakdown of collagen/gelatine may form a significant amount of cGP. The major composition of a soft gel capsule that is used to encapsulate blackcurrant concentrate comprising cGP is gelatine.

Preliminary studies have shown that the formation of cGP from gelatine breakdown may be dependent on lower pH and higher temperature. For example, the blackcurrant concentrate has a low pH of 2.5 which may breakdown the gelatine and may form cGP especially in a hot environment. The inventor has observed an increase of cGP concentration in the blackcurrant concentration after been capsulised in soft gel capsules (from 0.1 ng/mg to 10 ng/mg). This is a significant discovery where the pH and temperature range may increase the cGP levels in storage of the extract for enhanced effect. This same phenomenon may be applicable to any cGP extract encapsulated in a soft gel capsule irrespective of whether it is sourced from plant, animal, marine and/or fungal origin. Incubated blackcurrant concentrate with collagen at 350 C for two weeks also increases the cGP concentration from 40 ng/mg to 120 ng/mg compare to the blended sample that incubated at 40 C for 2 weeks. This phenomenon may applicable to other ingredients from plant, animal, marine and fungal origin.

The cGP extracts have not been used in the known and measurable manner now possible and as described herein. Further testing the efficacy of cGP using collagen blends in cognitive function of elderly people and metabolic disorders may exhibit a new or at least alternative use for cGP extracts that may provide significant and measurable benefit to patients potentially also without interfering with existing art treatments or causing unwanted side effects.

With respect to the described methods of measuring the changes in cGP and/or addressing neurological conditions, an advantage of the clinical application of cyclic glycine-proline (cGP) as a reliable plasma biomarker allows for selection of suitable patients for treatment and individual dosage regimes. The increase of cGP indicates the risk of a neurological condition and the reduction of cGP indicates the stage of the neurological condition. Advantageously, suitable patients for treatment may be selected based on their cGP level and the changes of cGP levels can be easily monitored for a tailored or personalised treatment dosage regime. Adjusting the dosage regime of the cGP extracts allows for effective treatment of the neurological conditions, thus improving long-term recovery (beyond 3 months), prevention of long time complications (cognitive impairment) associated with neurological conditions and peripheral neurological conditions. A further advantage is that cGP levels can simply be monitored by measuring urine samples for ease of analysis and critical for large scale clinical trials of cGP extracts in the future.

Further, with respect to natural products and associated bioactive compounds generally, the methods and uses described herein provide unique ways to know when it is appropriate to intervene with a treatment and further, provide ways to measure the success or otherwise of a treatment. This insight into timing and efficacy is something many pharmaceutical drugs would aspire to and not something commonly observed in natural products containing bioactive compounds.

The embodiments described above may also be said broadly to consist in the parts, elements and features referred to or indicated in the specification of the application, individually or collectively, and any or all combinations of any two or more said parts, elements or features.

Further, where specific integers are mentioned herein which have known equivalents in the art to which the embodiments relate, such known equivalents are deemed to be incorporated herein as of individually set forth.

### WORKING EXAMPLES

The above described methods relating to the increased concentration of cyclic glycine-proline (cGP) biomarker for prediction of risk and recovery of non-neurological and neurological conditions and the use of a cGP containing animal, marine and/or fungal based material such as a concentrate/extract of cGP for the treatment of same are now described by reference to specific examples.

### Example 1

### Identification of cGP Concentration in Animal, Fungal and Marine Sources

The inventor has previously identified the presence of cGP in nature plants including blackcurrant. However, the cGP concentration is low (approx. 0.1 to 1 ng/mg). The clinical application of plant cGP alone is insufficient for supporting and managing general health, especially ageing health. A source with increased concentration of cGP would be more beneficial for its clinical application in disease management. In particular, the management of acute conditions, like traumatic brain injury (TBI), stroke and other type of injuries where the supplementation of high concentration cGP products will help body reach the effective concentration faster.

Utilising novel processing/extraction methods, the inventor has identified a high concentration of cGP in the collagen from the animal and marine tissues. The extract is produced by maceration and followed by drying to form a concentrated powder that is then be micronized. The micronized powder may optionally be blended with other cGP containing ingredients or encapsulated if a capsule form is desired or otherwise processed to a final formulation.

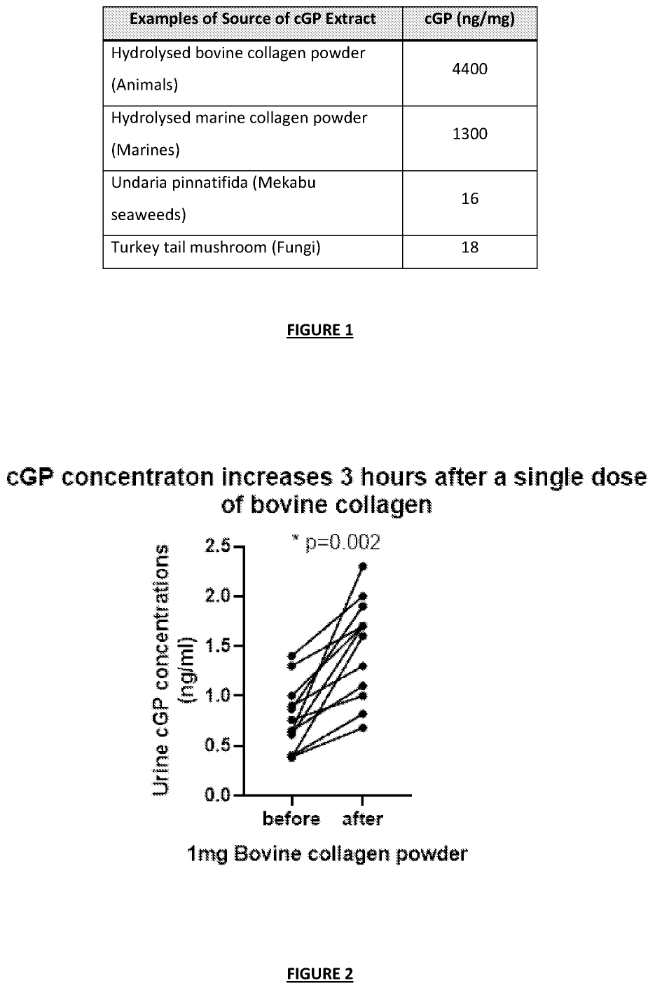

The cGP concentration in animal and marine sources is shown in FIG. 1. The concentrations of cGP are between 1300-4400 ng/mg which is thousand times higher than the counterpart plant-based products. As aforementioned, this higher concentration of cGP is essential for the management or supporting the management of acute conditions, like traumatic brain injury (TBI), stroke and other type of injuries.

To produce vegetarian or vegan version of cGPMAX™ the inventor has identified cGP concentrated ingredients sourced from mushrooms and seaweeds. Some examples are listed in Table 1. The health claim on brain function, for example the Turkey tail mushrooms has been claimed broadly, but no connections with cGP nor IGF-1.

### Example 2

### Pharmacokinetics of Oral Administration of Bovine Collagen Powder

A trial was completed by the inventor to see if supplementation of bovine collagen powder is 1) orally bioavailable, 2) supplementation of >40 times higher concentration of cGP derived from bovine collagen can lead to further increase of concentration cGP in human and 3) whether higher dose of cGP would assist the body to reach an effective concentration faster relative to supplementation of plant based products.

As part of their dietary intake, all the participants of the trial had been regularly taking cGPMax™ for more than a year. The concentration of cGP in cGPMax™ is minimal 10,000 ng/day (10 μg/day). The concentration of cGP can be different and manipulated through formulation based on the needs of clinical applications.

To determine whether supplementing the bovine collagen would further increase the cGP concentration in their body, an open label pharmacokinetics trial was conducted. The bovine collagen powder was dissolved in warm water. Urine samples were taken before and 3 hours after oral administration of a single dose of 100 mg. Paired t-test was used to analyse the difference between two time points.

FIG. 2 shows that the cGP concentration in urine is significantly increased 3 hours after oral consumption of 100 mg bovine collagen powder.

The protein binding plays a key role in the pharmacokinetics of cGP and cGP analogues. The ‘effective’ dose of cGP (or cGP analogues) was determined by the amount of cGP needs to saturate the protein binding site. This can be achieved through a shorter period administration of higher doses (as shown in FIG. 2) or a longer period of administration of lower dose cGP.

In comparison to plant based cGP products, the animal collagen can provide the effective dose of cGP in a shorter period of time. Collagen is orally bioavailable and the administration of 440 μg cGP (100 mg bovine collage powder) further increased the cGP concentration 3 hours after a single dosing in the participants. Thus, suggesting a rapid elevation of cGP level by collagen.

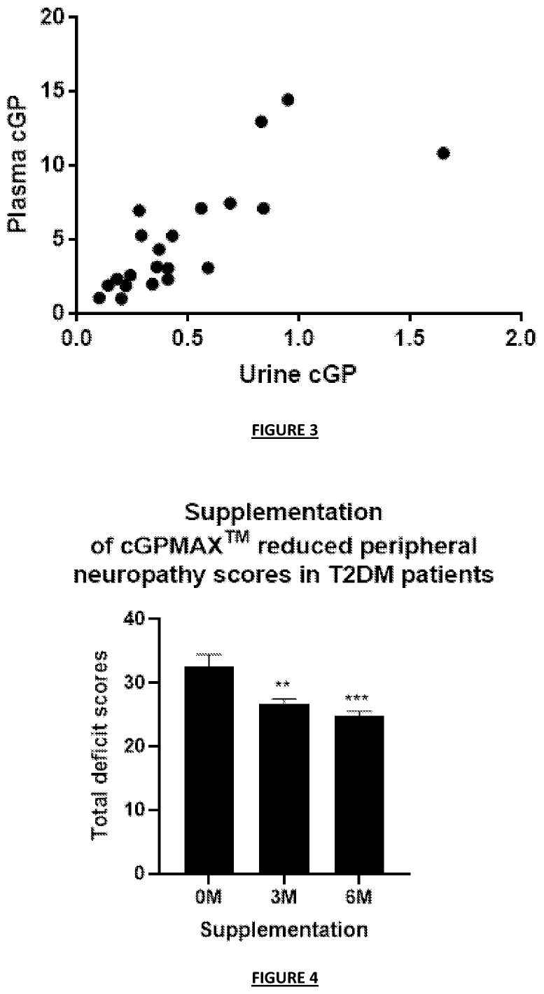

The concentration of cGP in urine is highly correlated to that in circulation, which had been assessed in patients with stroke. Pearson correlation analysis suggests a strongly positive correlation between urine and plasma cGP concentrations (r=0.78, p<0.001, FIG. 3).

The high correlation of cGP concentration between the urine and plasma also supports that the cGP is mainly excreted through the kidney. Any excessive amount of cGP in circulation will leave the circulation through the kidney. The pharmaceutically developed cGP analogues are used between high μg to low mg dose range.

In comparison, the cGP concentration provided by collagen supplementation is still lower than pharmaceutically developed synthetic analogues. Thus, the collagen-based cGP is safe for long-term supplementation. Hence, the absorption and tissues utilisation of cGP in nature foods may have advantages compare to the synthetic analogues.

Collagen contains high sequences of proline and glycine. Although it has a variety of cyclised prolines, there is no teaching or suggestion in the art that collagen contains cGP, and only has been suggested that the health benefit of collagen contributes mainly in skin and joints. The inventor for the first time has identified the presence of cGP in collagen. A large amount of experimental and clinical research outcomes from the inventor has demonstrated the association of clinical outcomes with endogenous cGP concentration, supported by the efficacy of cGP treatment in experimental studies. Such high concentration of cGP found in collagen is a significant mechanism in the health claim of collagen and other cGP derived sources. Hence, this discovery can extend the previous art beneficial claims of collagen from skin and joint health to cerebral and systemic vascular conditions, neurological, metabolic disorders and beyond.

Furthermore, the inventor has noted that fungi contains a variety of cyclic proline containing peptides, which may contribute to the health claims of fungi. However, there are currently no literature reports on the presence of cGP in fungi. Further testing in progress may identify the presence of cGP in various fungi, which may be a factor of contributing to the health claims of fungi. For example, the inventor has identified the high cGP concentration in the extract of Turkey tail mushroom (18 ng/mg) and several other mushrooms (Table 1).

### Example 4

Supplementation of cGPMAX™ Improved Peripheral Neuropathy in Patients with Type 2 Diabetes.

The inventor examined the efficacy of cGPMAX™ that is reformulated with hydrolysed collagen for peripheral neurological conditions. Type 2 diabetic mellitus (T2DM) is a major cause of peripheral neuropathy, a progressive neurological condition, which occurs in 25-50% of T2DM patients.

The initial stage of diabetic peripheral neuropathy (DPN) may present as sensory alterations that often progresses to sensory loss, numbness, pain, or burning sensations of the extremities, distal weakness, or atrophy, leading to impaired balance and gait. With no cure, the prognosis of peripheral neuropathy is poor and is a major cause of limb amputations. The 5 years survival rates of limb amputation are approximately 2 years and shorter compared with the colon, breast and prostate cancers.

An open label trial was carried out in the patients with >5 years clinical diagnosis of T2DM. The participants were scheduled for three hospital visits at various time points prior to the supplementation, 3 and 6 months after the supplementation. Eighteen T2DM patients had completed the follow-up. The daily supplementation of cGPMAX™ provided 20-40 μg of natural cGP. The peripheral neuropathy was assessed in both feet.

The sensory function to pressure, vibration and temperature were scored using Semmes-Weinstein monofilament test, Vibration perception threshold tests and cold/ward detection thresholds tests respectively, scored as: 1=normal; 2=reduced sensation and 3=no sensation. A total score>22 indicates abnormal sensory function and the higher the scores the more severe the deficit. Total scores of sensory to pressure, temperature and vibration were used for statistical analysis using repeated measures ANOVA.

All participants experienced an improvement in DNP symptoms, with the majority of them reported a noticeable improvement after the second month (or earlier) of supplementation.

Repeated Measures ANOVA showed a significant difference between the time points (p=0.0006, f[1.3, 22.25]=13.54, n=18). Compared to the baseline (before supplementation), the supplementation of cGPMAX™ shows a significant reduction in total DNP scores at 3 months (p=0.003) and 6 months (p=0.0007) best seen in FIG. 4.

This clinical outcome is the first evidence supporting the efficacy of cGPMAX™ in peripheral neurological conditions.

### SUMMARY/CONCLUSION

Animal, marine and fungal products have much higher cGP concentration than that of plant derived cGP extracts such as blackcurrant;
Oral administration of bovine collagen can further increase the cGP concentration in a human system (urine) of participants who had been using plant based cGPMAX™ long-term;
The significantly higher cGP concentration of animal, marine and fungal derived products can increase the cGP concentration to efficacious levels much faster than plant based sourced cGP. Given the formulation of plant-based cGPMax™ is primarily used for supporting health of normal people, new products from animal, marine and fungal derivatives may be developed for improving stroke recovery and other medication conditions with systemic and cerebral vascular conditions;
cGPMax™ that is formulated with collagen provided higher doses of cGP (20-40 μg/day). Supplementation of 20-40 μg/day cGPMAX™ for 6 months improved sensory function in T2DM patients with peripheral neuropathy; and
Any newly developed products also may be suitable for acute conditions that have increased demand for cGP in their system. For example, traumatic brain injury and on-set of stroke.

### Example 4

Increased cGP Concentration from the Chemical Breakdown of Collagen/Gelatine

The inventor also has found that collagen and gelatine (the heated form of collagen) is high in glycine-hydroxyproline amino acid sequences and unexpectedly the breakdown of collagen/gelatine forms a significant amount of cGP. The major composition of a soft gel capsule that is used to encapsulate blackcurrant concentrate comprising cGP is gelatine.

Preliminary studies have shown that the formation of cGP from gelatine breakdown is dependent on lower pH and higher temperature. Without being bound by theory, the blackcurrant concentrate (a plant based extract comprising cGP for experimental purposes) has a low pH of 2.5 which breakdowns the gelatine and forms cGP especially in a hot environment.

The inventor has observed an increase of cGP concentration in the blackcurrant concentration after been capsulised in soft gel capsules (from 0.1 ng/mg to 10 ng/mg). This is a significant discovery where the pH and temperature range may increase the cGP levels in storage of the extract for enhanced effect.

A stability study that stored a mixture of blackcurrant concentrate and collagen together at 35 degrees for 2 weeks increased cGP concentration to 110 ng/mg compared to that being stored at 4 degrees (40 ng/mg) in two weeks. A laboratory report of this stability study is available.

An empty soft gel (without any blackcurrant concentrate) or blackcurrant without being capsulised separately was evaluated.

The total amount of cGP in the soft gel alone is 26 ng/mg and blackcurrant concentrate is 0.1 ng/mg and the total amount of cGP is 7030 ng/capsule. However, upon analysis of an entire capsule (soft gel and encapsulated blackcurrant concentrate together, the total amount of cGP increased from 7030 ng cGP to 11000 ng. A report from an independent testing laboratory also has confirmed this cGP concentration.

The data suggests that the interaction between blackcurrant concentrate and gelatine soft gel is the key to increase the cGP from the initial levels, in which low pH, increased temperature and/or or other ingredients may have been involved in actively breaking down the gelatine in the soft gel. Further experimental tests are being conducted.

Aspects of methods relating to the clinical application of cyclic glycine-proline (cGP) biomarker for prediction of risk and recovery of non-neurological and/or neurological conditions with IGF-1 dysfunction and the use of a cGP containing animal, marine and/or fungal based materials such as concentrate/extract of hydrolysed bovine and marine collagen, mushroom and seaweed along with cGP encapsulated in soft gel capsules for the treatment of same have been described by way of example only and it should be appreciated that modifications and additions may be made thereto without departing from the scope of the claims herein.

What is claimed is:

1. A method of treating non-neurological and/or neurological conditions associated with IGF-1 dysfunction in an animal comprising the steps of:
a) obtaining a biological specimen from the animal;
b) measuring a concentration of cyclic glycine-proline (cGP) as a plasma biomarker for active concentration dependent insulin-like growth factor 1 (IGF-1) bioavailability in the biological specimen;
c) comparing either the measured cGP concentration and/or molar ratio of cGP concentration to total measured amount of IGF-1 in the biological specimen to a standard to confirm whether or not, in a continuum of results, the measured cGP concentration and/or ratio of cGP concentration to total measured amount of IGF-1 conforms to the relative standard for estimating IGF-1 function of the individual; and
d) administering a therapeutically effective amount of a concentrate/extract of derived from animal, marine and/or fungal based material to the animal to:
prevent a decrease in concentration of cGP and/or cGP to total measured IGF-1 molar ratio in an animal;
and/or maintain a pre-existing concentration of cGP and/or cGP to total measured IGF-1 molar ratio in an animal; and/or
increase the concentration of cGP and/or cGP to total measured IGF-1 molar ratio in an animal.

2. A method of predicting a risk of a non-neurological and/or neurological condition with age in an animal utilising cyclic glycine-proline (cGP) as a plasma biomarker with altered IGF-1 function comprising the steps of:
a) obtaining a biological specimen from the animal;
b) measuring a concentration of cyclic glycine-proline (cGP) as a plasma biomarker for active concentration dependent insulin-like growth factor 1 (IGF-1) bioavailability in the biological specimen at a first set age of the animal, or an initial stage of the non-neurological and/or neurological condition, or before treatment of an therapeutically effective amount of a concentrate/extract of cGP derived from animal, marine and/or fungal based material to the animal;
c) re-measuring the concentration of cyclic glycine-proline (cGP) plasma biomarker for active concentration dependent insulin-like growth factor 1 (IGF-1) bioavailability in the biological specimen at further set age intervals of the animal or further stage of the non-neurological and/or neurological condition, or after treatment of the therapeutically effective amount of the concentrate/extract of cGP derived from animal, marine and/or fungal based material to the animal; and
d) comparing either the measured cGP concentration and/or molar ratio of cGP concentration to total measured amount of IGF-1 in the biological specimen at the set age intervals relative to the first set age, or the initial stage of the non-neurological and/or neurological condition with IGF-1 dysfunction, or before the treatment of the therapeutically effective amount of a concentrate/extract of the cGP derived from animal, marine and/or fungal based material to the animal, in a continuum of results, to confirm whether or not there is a change in the measured cGP concentration and/or molar ratio of cGP concentration to total measured amount of IGF-1 thereby determining whether the animal is at an increased risk of developing a non-neurological and/or neurological condition from cognitive decline relative to a standard set of baseline data, and wherein the above measured ratio is used to select individual patients for cGP treatment and a suitable dosage for the cGP treatment therein.

3. A method of predicting the spontaneous recovery of an animal with a non-neurological and/or neurological conditions with IGF-1 dysfunction utilising cyclic glycine-proline (cGP) and cGP/IGF-1 molar ratio as the plasma biomarker for IGF-1 function comprising the steps of:
a) obtaining a biological specimen from the animal;
b) measuring a concentration of cyclic glycine-proline (cGP) plasma biomarker and active concentration dependent insulin-like growth factor 1 (IGF-1) bioavailability in the biological specimen at a baseline of the animal from onset of the non-neurological and/or neurological condition (<72 h in acute conditions);
c) re-measuring the concentration of cyclic glycine-proline (cGP) plasma biomarker and active concentration dependent insulin-like growth factor 1 (IGF-1) bioavailability in the biological specimen at further regular intervals of the animal during recovery;
d) evaluating functional recovery of the animal from the baseline and at further set intervals, and wherein the baseline concentration of CGP from a continuum of data predicts the short term outcome of non-neurological and/or neurological condition recovery of the animal such that a greater baseline cGP concentration, the more positive prognosis for the animal based on the evaluation of functional recovery.

4. The use of a concentrate/extract of cGP derived from animal, marine and/or fungal based material in the manufacture of a medicament formulated to:
prevent a decrease in concentration of cGP and/or cGP to total measured IGF-1 molar ratio in an animal; and/or
maintain a pre-existing concentration of cGP and/or cGP to total measured IGF-1 molar ratio in an animal; and/or
increase the concentration of cGP and/or cGP to total measured IGF-1 ratio in an animal.

5. The use of a concentrate/extract of cGP derived from animal, marine and/or fungal based material in the manufacture of a medicament formulated for oral administration to ameliorate the effects of and/or treat non-neurological and/or neurological conditions in a patient in need thereof.

6. A concentrate/extract comprising a therapeutically effective amount of cGP derived from animal, marine and/or fungal based material formulated for administration to an animal to:
prevent a decrease in concentration of cGP and/or cGP to total measured IGF-1 molar ratio in an animal; and/or
maintain a pre-existing (normal/physiological) concentration of cGP and/or cGP to total measured IGF-1 molar ratio in an animal; and/or
increase or to normalise the concentration of cGP and/or cGP to total measured IGF-1 molar ratio in an animal.

7. A method for ameliorating the effects of hypertension and/or a stroke; and/or treating hypertension and/or stroke; and/or reducing the symptoms associated with hypertension and/or stroke in a patient in need thereof, wherein the method comprises administering a therapeutically effective amount of a concentrate/extract of cGP derived from animal, marine and/or fungal based material.

8. A method for ameliorating the effects of and/or treating Parkinson's disease or the symptoms associated with Parkinson's disease, or complications associated with cognitive impairment in a patient in need thereof, wherein the method comprises administering a therapeutically effective amount of a concentrate/extract of cGP derived from animal, marine and/or fungal based material.

9. The method as claimed in any one of claims 1 to 3, wherein the non-neurological and/or neurological conditions or diseases are a Cerebrovascular accident or stroke, Mild Cognitive Impairment (MCI), Alzheimer's, vascular dementia, Rett syndrome, concussion, hypertension and its associated brain complications, Parkinson's and/or any other ageing related conditions or IGF-1 deficiency related conditions.

10. The method as claimed in any one of claims 1 to 3, wherein administration halts a decrease in cGP concentration as a result of a disease or condition.

11. The method as claimed in any one of claims 1 to 3, wherein administration increases cGP concentration by at least 1% above what would be measured in the patient with no cGP extract administration.

12. The method as claimed in any one of claims 1 to 3, wherein the animal is human.

13. The method as claimed in any one of claims 1 to 3, wherein the animal is healthy.

14. The method as claimed in any one of claims 1 to 3, wherein the animal has a pre-existing condition or disease state.

15. The method as claimed in any one of claims 1 to 3, wherein a standard or baseline is based on a set of data collected for a patient.

16. The method as claimed in any one of claims 1 to 3, wherein the standard or baseline is based on a set of data collected for a population.

17. The method as claimed in any one of claims 1 to 3, wherein the medicament is formulated for oral administration.

18. The method as claimed in any one of claims 1 to 3, wherein the medicament is formulated for parenteral administration.

19. The method as claimed in any one of claims 1 to 3, wherein the medicament is formulated as a pill, tablet, capsule, liquid, powder, micronized powder, gel, soft gel full of liquid and/or combinations thereof.

20. The method as claimed in claim 19, wherein the micronized powder particle sizes substantially between 1 to 1000 micron.

21. The method as claimed in any one of claims 1 to 3, wherein the medicament is administered to provide a therapeutically effective dose of concentrated cGP of at least between 10,000 to 100,000 ng (10-100 μg) as daily dose.

22. The method as claimed in any one of claims 1 to 3, wherein there is at least a 25% increase of cGP in the cerebrospinal fluid (CSF) after cGP extract supplementation.

23. The concentrate/extract as claimed in any one of claims 4 to 6, wherein the concentrate/extract with high concentration of cGP is derived from any animal, marine and/or fungal sources including any living organism selected from any of bovine, marine and bone collagen sources.

24. The concentrate/extract as claimed in any one of claims 4 to 6, wherein a hydrolysation process of the extract cleaves collagen/gelatine proteins and forms linear peptides with repeated sequences of hydrolysed proline-glycine, wherein cyclisation of these linear peptides leads to production of cGP to normalise IGF-1 function.

25. The concentrate/extract as a claimed in claim 23, wherein the concentrate/extract is hydrolysed bovine collagen powder, collagen marine skin powder, hydrolysed fish skin powder and/or bone collagen.

26. The concentrate/extract as claimed in any one of claims 4 to 6, wherein the concentrate/extract is processed and produced using body parts selected from: skins, proteins, bones, muscles tendons and the like and/or combinations thereof.

27. The concentrate/extract as claimed in claim 26, wherein the concentrate/extract is produced by maceration of animal, marine and/or fungal material, followed by drying to form a concentrated powder that is micronized.

28. The concentrate/extract as claimed in claim 27, wherein the micronized powder is encapsulated within soft gels and/or hard shells.

29. The extract as claimed in any one of claims 4 to 6, wherein the concentrate/extract blend comprises Cyclic glycine-proline (cGP) in a concentration to provide at least 10,000 ng or more as a daily dose.

30. The concentrate/extract as claimed in any one of claims 4 to 6, wherein the concentrate/extract, either in combination or separately is attributed to the observed effect in vivo on the binding of IGF-1 and the observed increase in cGP concentration and/or cGP to total IGF-1 ratio.

31. The concentrate/extract as claimed in any one of claims 4 to 6, wherein the concentrate/extract comprises other bioactive compounds besides cGP.

32. The concentrate/extract as claimed in any one of claims 4 to 6, wherein, collagen and gelatine (the heated form of collagen) is high in glycine-hydroxyproline amino acid sequences and wherein the breakdown of collagen/gelatine forms a significant amount of cGP.

33. The concentrate/extract as claimed in claim 32, wherein formation of cGP from gelatine breakdown is dependent on lower pH and higher temperature.

34. The concentrate/extract as claimed in claim 32 or claim 33, wherein, an increase of cGP concentration following capsulisation in soft gel capsules is from 0.1 ng/mg to 10 ng/mg.

35. The concentrate/extract as claimed in any one of claims 32 to 34, wherein incubated concentrate with collagen at 350 C for two weeks increases the cGP concentration from 40 ng/mg to 120 ng/mg.

36. A soft gel or hard shell capsule used to encapsulate cGP extracted material for administration thereof, and wherein the cGP concentration of the extracted material contained therein increases over a period of time.
